(19) United States
(12) Patent Application Publication
(10) Pub. No.: US 2010/0191163 A1
(43) Pub. Date: Jul. 29, 2010

(54) Dynamic-response, anatomical bandaging system and methodology

(75) Inventors: Michael R. Dennis, St. Helens, OR (US); Casey A. Dennis, Sequim, WA (US)

Correspondence Address: ROBERT D. VARITZ, P.C., 4915 SE 33RD PLACE, PORTLAND, OR, 97202, US

(73) Assignee: MJD Innovations, L.L.C.

(21) Appl. No.: 12/657,570

(22) Filed: Jan. 21, 2010

Related U.S. Application Data
(60) Provisional application No. 61/268,934, filed on Jun. 18, 2009.
(60) Provisional application No. 61/206,127, filed on Jan. 28, 2009.

(51) Int. Cl. A61F 5/05; A61F 13/02

(52) U.S. Cl. 602/5; 602/46

(57) ABSTRACT

A dynamic-response anatomical bandaging system and methodology utilizing a limb-wrappable, layered, dynamic-response, bandaging expanse which includes a dynamic-response, pressure-applying layer displaying a compressive-load versus compression-deflection behavior which is characterized by a curve having a substantially linear region in which a major change in compression deflection relates to an anatomically insignificant change in compressive load. The system and methodology also feature, relative to use of the bandaging expanse, freely attachable and detachable, dynamic-response (a) splinting structure, and (b) expanse-edge-overlap wrap-closure tensioning structure.

### CROSS REFERENCES TO RELATED APPLICATIONS

This application claims filing-date priority to two, different U.S. Provisional Patent Application Ser. No. 61/268,934, filed Jun. 18, 2009, for “Bandaging/Splinting Structure and Methodology”, and Ser. No. 61/206,127, filed Jan. 28, 2009, for “Bandaging/Splinting Structure and Methodology”. The entire disclosure contents of each of these two, prior-filed, currently co-pending U.S. Provisional Patent Applications are hereby incorporated herein by reference.

### BACKGROUND AND SUMMARY OF THE INVENTION

This invention relates to an anatomical bandaging system and methodology, and in particular to dynamic-response bandaging for the human anatomy. The invention proposes a unique structural system, and an associated methodology, which are based upon applying, automatically, stably and self-adjustably, throughout the entirety of a bandaged region, such as around a limb like the leg, and due entirely to the dynamically responsive structural natures of the main components making up the system, a substantially constant, dynamically-maintained, evenized pressure to such a region.

Dynamic-response bandaging, and the respective dynamic-response behaviors of the main components in the proposed system, as the term “dynamic-response” is used herein, involves the specific (a) compression, (b) bending/flexing, and (c) elastomeric stretching and relaxing, respective motion-response characteristics of the three, principal structural elements of the invention which include—(a) a layered bandaging expanse, (b) a flexible splinting structure (referred to herein as a bandaging-expanse-cooperative splinting structure, which may be singular in splinting-component nature, or composite by virtue of including plural splinting components), and (c) what is termed herein a wrap-closure tensioning structure that functions, with the expanse wrapped around a limb with appropriate edges overlapped, to hold the wrap in place under tension (as will be explained).

While those skilled in the medical arts recognize that there are many important considerations to be taken into account with respect to anatomical bandaging, in addition to the significant consideration involving preventing moisture buildup in the confined interface between bandaging and the anatomy, another extremely important consideration is that, ideally, bandaging pressure applied to the anatomy should have a special characteristic. More specifically, it should be greater than the pressure of static fluid in the bandaged portion of a limb (one range limit), in order to control, i.e., suppress and/or prevent undesirable, fluid-retention swelling, and it should also be less than that pressure which would undesirably block venus-return blood flow in a bandaged limb portion (a second range limit). Bandaging pressures established or left lying outside of this important range can cause serious problems. Effective bandaging pressure changes involving pressures, such as locally occurring pressure changes that occur due to one reason or another, lying within these two limits are considered herein to be “anatomically insignificant”. A useful pressure range to think about in regard to this matter of anatomical insignificance is between about 0.3- to about 0.7-psi.

Ideally, a correctly initially “set” bandaging pressure should hold as a substantially constant value throughout a bandaging period, but conventional approaches do not readily accomplish this, and it is usually the case that a “bandaging attendant” is not poised to correct, let alone easily to recognize, an unacceptable, unknowingly-developed, “out-of-range” pressure condition before difficulties, and sometimes very serious ones, step into play.

In this context, those skilled in the relevant art are certainly familiar with applying conventional bandaging to the anatomy, and normally fully understand the above considerations. Accordingly, with respect to freshly-applied bandaging pressure, these persons generally have the requisite skills to assure that at least an initially placed bandaging structure will, at the outset of a bandaging time period, meet the important, above-described, anatomical-applied-pressure, range conditions. However, experience has shown that, unfortunately, such initially created, ideal compression pressure conditions on the surface of the anatomy change, and sometimes change dramatically, without giving any “warning” to an outside observer that an undesirable bandaging pressure condition exists. Such a pressure change can come about in a number of different ways, and often in a very localized region within a bandaging environment. For examples, various anatomical motions may alter bandaging pressure. Blood pulsatile activity may introduce changes. Fluid-based swelling is also a frequent culprit. There are others.

The present invention directly addresses these important, and other, considerations relative to anatomical bandaging. As mentioned above, all three of the main components, or elements, of the invention feature, by virtue of their specific internal constructions, a respective, dynamic response behavior which addresses and aids desired range control over anatomical bandaging pressure.

There are many applications which those skilled in the art will recognize as being suitable for useful implementation of the present invention. Some of these applications include (a) wrapping and stabilizing a wound just received (as in an accident), and during the transport of a wounded party to a medical facility, (b) the bandaging of a post-surgery wound, (c) the compression bandaging of other kinds of post-medical-treatment, but not necessarily surgery-related or wound-related conditions, (d) the wrapping and stabilizing, as by splinting, of a limb to deal with something such as a broken bone, and (e) many others.

From the above background discussion, it will be clear that one of the important aspects of the present invention involves the manner in which pressure is applied to, and maintained as constant as possible over time in, a bandaged/wrapped anatomical area. With this in mind, chosen, in accordance with the present invention, to apply dynamically (pressure-range) controlled, evenized pressure over a wrapped, bandaged area is a special, plural-layered bandaging expanse which features a low-rebound, viscoelastic, acceleration-rate-sensitive cushioning foam layer having, very importantly, a selected, internal structural character that exhibits a compressive-deflection vs. compression-force curve which includes an extremely linear region over which a relatively wide change in compressive deflection is accompanied by an anatomically insignificant change in applied bandaging pressure. With such a cushioning material utilized to apply “bandaging” compression, and with an appropriate, initial, anatomical-compression level established in the “bandaged” area, anatomical motions of almost any character, as well as anatomical protrusions, such as bone protrusions, static-fluid induced swelling, and various pulsatile activities, among other things operative in the bandaged environment, by our many, careful observations, do not change the fact that the pressure applied over the entire area (a) is substantially the same everywhere, with that pressure (b) remaining in a proper pressure range substantially unchanged notwithstanding occurrences of the various kinds of “pressure disturbances” mentioned above.

The outer side, or surface, of the proposed bandaging expanse is furnished preferably with the pile-portion of conventional hook-and-pile fastening structure.

Also featured by the structure and methodology of the present invention, where appropriate in a bandaging situation, is dynamic-response splinting which is based upon quick and easy hook-and-pile application of a stiffening splinting component, or splint, appropriately shaped with a body which is preferably relatively thin and blade-like in nature, against the “pile-portion” outside surface of the mentioned bandaging expanse. The splint body which is designed, when used, to lie “somewhat flat” against the outer side of the bandaging expanse, is flexible, and resiliently bendable about many different, preferential, in-plane bending axes, and dynamic-response flexing about one or more of these axes, in relation, for example, to anatomical motion, cooperates with the associated bandaging expanse, and aids in maintaining consistency of evenized, bandaging-pressure application by that expanse.

Another, related, splinting feature of the invention involves the implementation of selectively differently shaped splinting components, including angularly profiled components, whose opposite sides are preferably prepared with the two, different, releasably matable components of hook-and-pile material, with the idea that an entire, overall “splint structure” may be formed as a composite structure including what might be thought of as plural, splinting segments that are quickly assembleable simply by hook-and-pile contacting of one to another.

All of the proposed forms of splinting components may, if desired, be applied, later removed, and then later reused.

Further proposed by the present invention is anatomical compression bandaging which, in one form, includes (a) a compression bandaging expanse possessing viscoelastic cushioning structure (as above described) which is wrappable in tension around an anatomical limb in what is referred to as an exposed-edge, edge-to-edge overlap manner, and (b) an attachable/removable, relatively short, ribbon-like, bridging closure (or tensioning) structure which does not encircle/circumsurround an expanse-wrapped limb, but rather simply crosses the edge-overlap region of the wrapped bandaging expanse. The bridging closure structure includes a pair of spaced-apart end attaching components that are designed for quick attachment and detachment to the pile-portion outside of the bandaging expanse through included hook-portion elements of hook-and-pile-type attaching mechanism. Extending between these two end components is an elongate elastomer-ribbon bridge which enables easy and quick user-adjustment of the tension which is developed in overall compression bandaging during use.

All tension-produced, wrapping-compression is originated, and initially developed and established, entirely within the elastomer material included in the short, bridging closure structure, which elastomer material is not, in the region between the end attaching components, affixed in any way to the outwardly exposed surface of the compression expanse. This arrangement uniquely allows the elastomer bridge—free from “direct” attachment to an associated bandaging expanse and its overlapping edges—to stretch and relax in a dynamic-response manner so as to accommodate various anatomical motions, or other “disturbances”, etc., which might otherwise challenge proper maintenance of consistent, evenized bandaging pressure.

During use, the elastomer in the bridging closure structure spans the exposed edge of the wrapped compression expanse in a manner, and with specific, smooth structure, which produces no damage, such as abrasion and/or snagging, to this edge. The full length of the elastomeric element, which preferably is formed from a non-latex material possessing a stretch capability of up to about 200% or so, is available for quick adjustment of the bridging structure throughout a relatively wide range of user-selectable tensions to define compression wrapping around an associated anatomical limb.

In practice, the systemic form of the invention, and as will be more fully explained below, may use one or more of the three, main dynamic-response components—always involving use of at least the described bandaging expanse.

These and various other features and advantages which are offered by the present invention will become apparent from the detailed description of the invention presented below, accompanied by a reading of the accompanying drawings.

### DESCRIPTIONS OF THE DRAWINGS

FIG. 1 is a simplified, anatomy-side view of a layered, dynamic-response, anatomical bandaging expanse made in accordance with a preferred and best-mode embodiment of the present invention. Portions of this expanse have been broken away to reveal details of construction.

FIG. 2 is an enlarged, fragmentary view taken generally along the line 2-2 in FIG. 1.

FIG. 3 is a graph illustrating five curves describing the respective compressive-load versus compression-deflection behaviors of five different, dynamic-response, viscoelastic foam materials that are employable satisfactorily as a particular one of the layers in the bandaging expanse of FIGS. 1 and 2. The central curve in this figure illustrates this characteristic for the preferred viscoelastic foam material which is employed.

FIG. 4 is a simplified plan view of the bandaging-attaching face or side of what is referred to herein as an elongate, expanse-edge-attachable/removable, dynamically-responsive, wrap-closure tensioning (or bridging closure) structure which is employed to fix in place, and to introduce wrapping tension into the bandaging expanse illustrated in FIGS. 1 and 2. This tensioning structure is used under circumstances where the bandaging expanse is applied as an edge-overlap wrap around an anatomical limb, such as around the leg.

In FIG. 4, the illustrated tensioning structure is shown in solid lines in a nominal, un-stretched condition, and in dashed, and dash-dot, lines, respectively, in two, differently stretched conditions—that which is illustrated in dashed lines picturing a lesser stretch than that which is pictured in dash-dot lines. Double-arrow-headed dashed and dash-dot lines, respectively, help to illustrate these two stretches.

FIG. 5 is an edge view taken generally along the line 5-5 in FIG. 4.

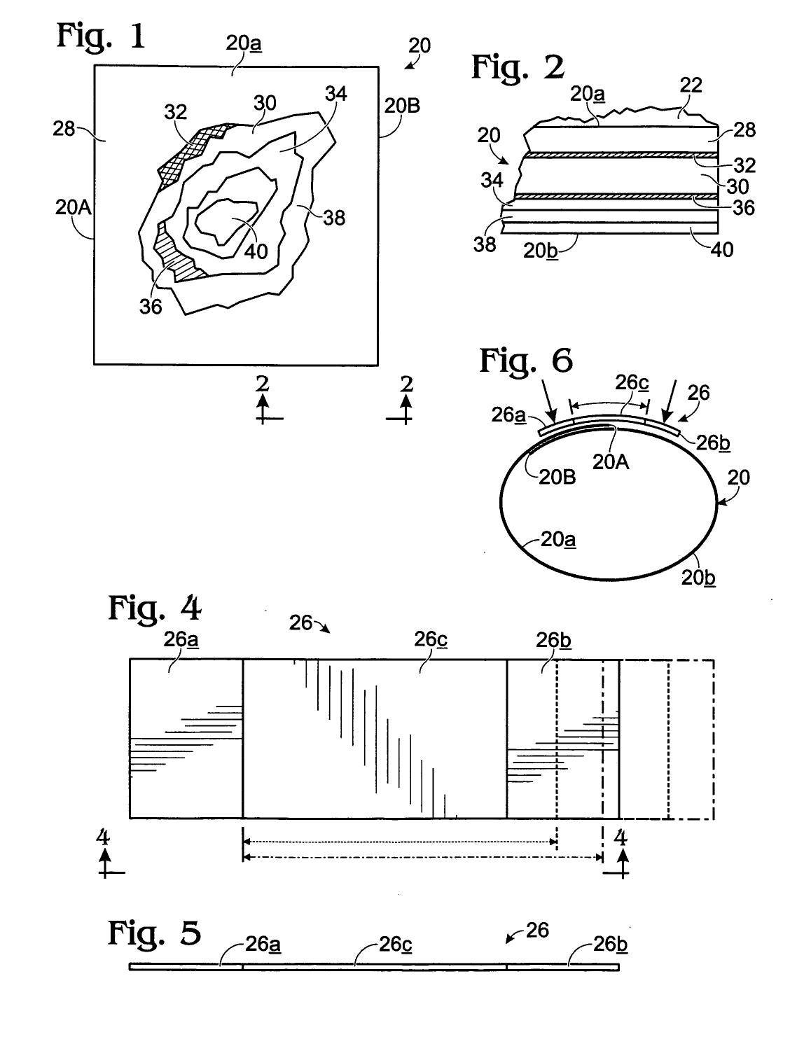

FIGS. 4 and 5 are drawn on about the same scale—one which is intermediate the scales employed in FIGS. 1 and 2.

FIG. 6 is a simplified view picturing the bandaging expanse of FIGS. 1 and 2 in an edge-overlap condition wrapped around a non-illustrated anatomical limb, and fixed in place, and put under appropriate tension, by a plurality (only one being shown) of tensioning structures like that shown in FIGS. 4 and 5. The bandaging expanse, and the single tensioning structure, shown in FIG. 6 are pictured, relative to one another, in a modestly exploded condition, with the two, single-headed, downwardly pointing arrows that appear in this figure representing hook-and-pile attachments between the opposite ends of the tensioning structure and the outer side, or surface, the wrapped expanse, and with slightly downwardly curved, double-headed arrow in this figure representing a tensed and stretched condition in the illustrated tensioning structure. FIG. 6 is drawn on approximately the same scale as that which is employed in FIG. 1.

FIG. 7, with certain illustration portions broken away to show details of construction, is a fragmentary, plan view of what is referred to herein as expanse-cooperative, dynamic-response, flexible splinting structure. FIG. 7 is drawn on about the same scale as that which is employed in FIG. 6.

FIG. 8 is a view taken generally along the line 8-8, in FIG. 7.

The three, different types of components that are pictured in FIGS. 1-8, inclusive, collectively make up the dynamic-response bandaging system of the present invention in its preferred and best-mode forms.

FIGS. 9 and 10 are, respectively, lateral and rear, fragmentary views illustrating all of the several components which are pictured in FIGS. 1-8, inclusive, applied collaboratively to a person's left leg, ankle and foot. FIGS. 9 and 10 are drawn on a scale which is slightly smaller than that which is employed in FIGS. 6-8, inclusive. In these two figures, bandaging illustration and specific descriptive discussion below focus principally on bandaging which is provided for the leg.

In all of the structural-illustration drawing figures herein, individual components, and portions thereof, are not necessarily drawn to scale with respect to one another. In some instances, sizes have been exaggerated so that certain things could more readily be seen at the drawing scales selected for the drawings.

### DETAILED DESCRIPTION OF THE INVENTION

Turning now to the drawings, indicated generally in an isolated fashion at 20 in FIGS. 1 and 2 is what is referred to herein as a layered, dynamic-response anatomical bandaging expanse having an inner side 20a, which is applicable directly to, and in contact with, the human anatomy, a portion of which anatomy is shown fragmentarily at 22 in FIG. 2, and an outer side 20b. Expanse 20 further includes a pair of spaced, opposite edges 20A, 20B, and, as will further be explained, is deployable in tension as and overlapping-edge (20A, 20B) wrap extending around an anatomical limb (such as the leg), in the manner generally shown in a very simplified form in FIG. 6 for the expanse. In order for FIG. 6 to present this wrapped condition of expanse 20 in as simple and uncluttered a form as possible, no anatomical limb, per se, is illustrated in this figure.

Bandaging expanse 20 forms one of three main components, or elements, of a dynamic-response bandaging system which is made in accordance with the structure of the present invention, the two other main components, or elements, in which system taking the forms, respectively, of what are referred to herein as (a) an expanse-cooperative, dynamic-response, flexible splinting structure (singular-component, or composite plural-component), shown generally at 24 in FIGS. 7-10, inclusive, and (b), an elongate, expanse-edge-attachable/removable, dynamically-responsive, wrap-closure tensioning structure 26, seen in FIGS. 4-6, inclusive, 9 and 10.

Bandaging expanse 20 herein is made up of five, joined, unified layers of different fabric and foam materials, and a pair of special, flexible, gas-permeable, moisture-resistant, non-latex adhesives. The five “fabric layers” include (1) a medical-grade, tricot, moisture-wicking fabric layer 28 (also heat-, friction- and shear-minimizing against the skin) which has an upper, anatomy-facing side in FIG. 2 that forms the previously mentioned inner side of expanse 20, (2) a dynamic-response, low-rebound, acceleration-rate-sensitive, anatomical-pressure-applying, viscoelastic foam layer 30 whose dynamic-response, cushioning-compression characteristics that are important in the functionality of the present invention will be described shortly, and which is bonded to layer 28 through one of the just-above-mentioned adhesive layers shown at 32, (3) a gas-permeable, moisture-resistant, abrasion-inhibiting fabric layer 34 which is joined to layer 30 through the other, above-mentioned, adhesive layer, here shown at 36, (4) a polyurethane foam layer 38 which is joined to layer 34, and (5) a fabric layer 40 referred to herein as a pile-portion fabric layer which takes the form of the pile portion of conventional hook-and-pile connection fabric material, such as the material referred to as Velcro®. this layer being joined to layer 38. The underside of layer 40 in FIG. 2 forms the previously mentioned outer side of expanse 20.

Each of the several, bandaging-expanse layers/materials just described is individually conventional in construction, readily commercially available, and is hereinbelow identified, in terms of specific, representative materials which we have preferred (others being usable as well), in the following manner. Moisture-wicking fabric layer 28, of which a number are generally known in the art is preferably the material identified as Orthowick™, made by Velcro USA, Manchester, N.H. The two, mentioned, flexible adhesive layers are alike, and preferably are formed of a glue made by Henkle, Inc, headquartered in Dusseldorf, Germany, and referred to as Imperial 1059 glue.

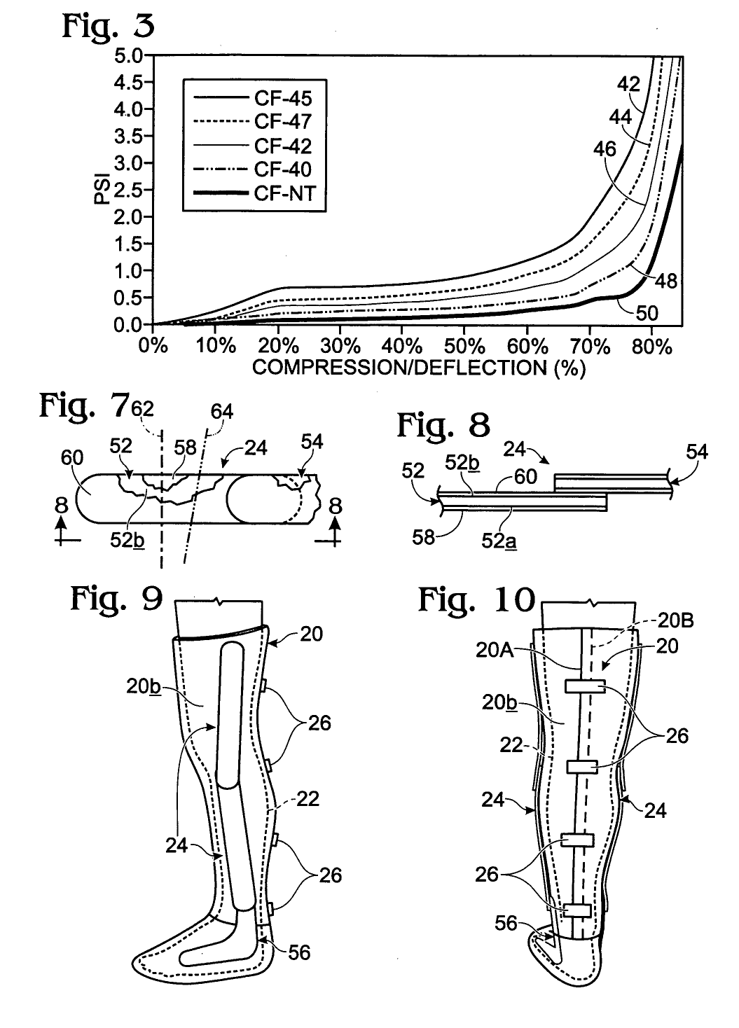

Dynamic-response foam layer 30 is formed of a temperature-, pressure-, and acceleration-rate-sensitive, cellular, viscoelastic foam material, and is preferably one of the several foam materials (CF-40, CF-42, CF-45, CF-47, CF-NT) sold under the trademark Confor®, and made by EAR Specialty Composites in Indianapolis, Ind. This layer, for which we have particularly chosen Product No. CF-42, has a preferred thickness for the purpose of the present invention, of about 0.375-inches, and, as do all five of the just-mentioned Confor® products, has a very special, internal, dynamic-reaction characteristic which will be more fully described shortly, and which is illustrated by the central one of the five curves appearing in FIG. 3 in the drawings. Each of these material-characteristic curves exhibits a compressive-load versus compression-deflection behavior having a large, substantially linear, central region in which a major change in compressive deflection, occurring within a range of about from 20% to about 60% compressive deflection (or about 0.15-inches in the preferred, layer-30 material thickness mentioned above), relates to what one can think of as being an anatomically insignificant change in associated compressive load, typically lying, as can be seen, within a total range approximately centered on about, 0.5-pounds-per-square-inch. As has been mentioned earlier herein, the overall, operative, compressive range which characterizes layer 30 in bandaging expanse 20 more specifically is between about 0.3-psi to about 0.7-psi.

Layers 34, 38, 40 herein preferably form portions of a commercially available, single, integrated material having an overall thickness of about 0.125-inches, and sold as the product referred to as Veltex®, made by Velcro USA, Inc. in Manchester, N.H.

The overall thickness of bandaging expanse 20 herein is preferably about 0.5-inches.

Continuing with a structural description relating to bandaging expanse 20, and focusing attention for a moment on the graphical presentation of FIG. 3, this figure shows at 42, 44, 46, 48, 50, five different curves illustrating compressive-load versus compression-deflection behavioral characteristics, respectively, of previously-mentioned viscoelastic foam materials CF-47, CF-45, CF-42, CF-40, CF-NT. As can be seen, it is central curve 46 which illustrates specifically this behavior of the viscoelastic foam material, CF-42, which has been chosen preferably for employment in previously mentioned foam layer 30 in expanse 20.

What is made clearly evident by the curves presented in FIG. 3 is that, with respect to each of the five, different, viscoelastic foam materials whose characteristics are pictured in this figure, each of these material's so-pictured compressive-load versus compression-deflection characteristic exhibits a relatively large (long), linear region that extends generally between, and from, about 20% compression deflection to about 60% compression deflection. In this context, and with specific regard to the so-illustrated behavioral characteristic of preferred material CF-42 shown by curve 46, between these two, percentage, linear-range-defining conditions, a major percentage change of around 40% total compression-deflection differential is associated with what has been described above as an anatomically insignificant change in compressive load. More specifically, and focusing on the data presented in curve 46, the compressive-load change which accompanies this large (about 40%) percentage deflection change varies only from about 0.3- to about 0.7-psi.

Experience has shown that when bandaging expanse 20 is properly applied by one of skill in the art as a wrap around an anatomical limb, such as around the leg, with suitable wrapping tension introduced into this expanse, and accordingly, a suitable level of surface compression applied to the anatomy, the observed condition of foam layer 30 in the expanse is such that this foam layer exhibits, under those conditions, a compression deflection of around 35% to about 40%. This condition is observably achieved in normal use of the bandaging expanse proposed by the present invention when a person of ordinary skill in the medical arts applies the bandaging expanse with what might be thought of as an entirely normal wrap-tension force. Observation also clearly is that when this is done, the compressive load applied to the anatomy nominally lies about centrally in the linear range of the characteristic for the employed viscoelastic foam material, and specifically, for the preferred material CF-42, exists at about, or slightly less than, 0.5-pounds-per-square-inch, a compressive pressure which fully meets the important objective mentioned earlier herein of applying a compressive anatomical force which is above that expected for normal static fluid pressure in the anatomy, but below that which would cause undesirable venus-return blood flow.

As has been mentioned, bandaging expanse 20 is intended to be employed preferably as a wrap around a portion of the anatomy, such as an anatomical limb like the leg. Expanse 20, as illustrated in FIG. 1, is shown herein as a rectangle, but it need not necessarily have this particular perimetral shape. For example, the bandaging expanse structure may be formed in large sheets or rolls from which specific perimetrally outlined shapes may be cut for use. It may also be completely preformed in different shapes.

It turns out that a perimetral shape for expanse 20 which works quite well for bandaging a limb like the leg is a rectangle like that which is shown in FIG. 1. When this expanse is correctly applied as a wrap, as is generally illustrated in FIG. 5, it is applied in an edge-overlap wrap manner. Thus, in FIG. 5, such an edge-overlap condition is clearly pictured, with edge 20A overlapping edge 20B preferably by about three 2-3-inches.

While what may be thought of as a “full content”, dynamic-response bandaging system will include all three of the main components described hereinabove, it is entirely possible, in a systemic sense, to implement in accordance with the invention a partial-component bandaging system by using one of (a) the bandaging expanse alone, (b) the bandaging expanse along only with the tensioning structure, or (c) the bandaging expanse along only with the splinting structure.

Accordingly, where the bandaging expanse is used completely by itself, it, under those circumstances, may be thought of as constituting the invented bandaging system, and may be held in place, and applied under tension to produce compression in the surface of the anatomy, by a conventional overwrap of something like a traditional Ace-bandaging ribbon. Where the bandaging expanse is used only with the proposed tensioning structure, it is, of course, the tensioning structure which functions to introduce tension into the wrapped expanse, and compression into the surface of the anatomy (a preferable situation). Where the bandaging expanse is employed only with the proposed splinting structure, tension in the wrap, and compression in the surface of the anatomy, may be created by an Ace-bandage-ribbon overwrap.

Turning attention now to the construction of tensioning structure 26, and focusing specifically on FIGS. 4 and 5, this structure has an elongate, thin, rectangular configuration, as pictured in these two drawing figures. Structure 26 includes three subcomponents, or portions, namely, a pair of spaced, opposite-end, hook-and-pile, hook-portion, fabric end components 26a, 26b, made of the material sold under the above-referred-to trademark Velcro®, joined, as by stitching, to a central, elongate, elastomeric bridge 26c. Bridge 26c may be formed of any suitable elastomeric material, and preferably one which has an elongation capability of up to about 200%.

The special operational advantages of the three-component structure just described for each tensioning structure 26 were discussed earlier herein.

Referring now to splinting structure 24 as seen in FIGS. 7 and 8, the main element within this structure takes the form of a flexible splint body, such as the two splint bodies shown at 52, 54 in these two figures. Each of these bodies possesses a thin, planar, blade-like configuration, formed of a material such as conventional ABS plastic, or aluminum, with a thickness of approximately 0.125-inches. An appropriate aluminum is conventionally available type 6064T3 flat-bar aluminum. The splint bodies in splinting structure 26, as mentioned earlier, are referred to herein as being dynamic-response components on account of their springy flexibility.

It will be apparent to those skilled in the art that the exact perimetral dimensions and shapes of the proposed splint bodies may be defined differently in accordance with the anatomical regions where splinting is desired as a part of the bandaging system of the present invention. For example, for a leg-bandaging application, such as the one illustrated in FIGS. 9 and 10, elongate linear splint bodies with widths of about 2-3-inches, and lengths of about 12-inches or more may be employed. In the context of utilizing a splinting structure with a quite differently shaped splint body on a differently shaped bandaging expanse, and considering the ankle-and-foot-including bandaging and splinting application pictured in FIGS. 9 and 10, a somewhat right-angular splinting structure, such as that shown at 56 in FIG. 9, may be employed.

In accordance with the present invention, each splint body possesses what is referred to herein as an inner side and an outer side. For above-mentioned splint body 52, the inner side thereof is shown at 52a, and the outer side at 52b.

Affixed to the inner side of each splint body is a hook-portion fabric of conventional hook-and-pile fastening material. Such a hook-portion material affixed to splint body side 52a is shown generally at 58. Affixed to the outer side of each splint body is a pile-portion fabric of conventional hook-and-file fastening material, such as the pile-portion of this material shown at 60 affixed to splint body side 52b. These hook and pile-portions of hook-and-pile fastening material enable plural splinting bodies effectively to be joined releasably to one another in an infinite variety of ways to form a composite splinting structure such as the composite splinting structures that are specifically illustrated in FIGS. 7-10, inclusive.

A final point to be made with respect to the splint bodies that make up the individual splitting-structure components is that these thin, blade-like bodies are characterized each with a plurality, indeed almost an infinity, of preferential, in-plane bending axes, like the two axes which are shown, respectively, by a dash-dot line 62, and by a dash-double-dot line 64, in FIG. 7. It will be apparent to those skilled in the art, given the structural natures of the described splint bodies, that these preferential, in-plane bending axes may effectively lie substantially anywhere within the splint bodies, depending upon how a user of the splinting structure of this invention chooses to apply splinting structure in a bandaging operation, and also how, once bandaging has been installed, anatomical motion and other motion disturbances may cause flexure/bending to occur.

From the various descriptions that have been given above regarding the several components which collectively make up the full dynamic-response bandaging system of the invention, it should be readily apparent how a bandaging operation, utilizing these components, may preferably be performed to create bandaging like that which is shown in FIGS. 9 and 10. For such an operation, one or more bandaging expanse(s), like expanse 20, appropriately shaped perimetrally is(are) wrapped to an edge-overlap condition, and then secured in place, and placed in tension to apply compression support to the wrapped anatomy, by use of a distribution, such as the distribution shown in FIGS. 9 and 10, of tensioning structures 26. If splinting is to take place, one or more of the hook-and-pile-equipped splint bodies is (are) applied easily and quickly both to one another, where composite splinting is required, and under all circumstances to the outer pile-portion surface of the applied bandaging expanse or expanses.

From the standpoint of the methodology which is proposed and offered by the present invention, and implemented at least in part by the several structural components discussed above, that methodology may be described as a dynamic-response anatomical bandaging method including (a) placing a dynamic-response, anatomical bandaging expanse as a wrap around a selected portion of an anatomical limb to form a wrapped portion of the limb, and (b) in relation to and as a consequence of such placing, applying, in accordance with self-compensating response occurring per se within the structure of the placed expanse, dynamically evenized wrap pressure to the wrapped portion of the limb, with such wrap pressure, under all dynamic circumstances with the expanse in place, exceeding that of static fluid pressure in the wrapped limb portion, but being less than that which would block venus-return blood flow in that limb portion.

In the practice of this methodology, the mentioned wrap pressure preferably lies in the range of about 0.3- to about 0.7-psi.

The proposed methodology further includes, before, and to accommodate, bandaging-expanse placing, providing a dynamic-response bandaging expanse which is characterized by including a dynamic-response, viscoelastic foam layer formed of a material which exhibits a compressive-load versus compression-deflection behavior characterized by a curve having a substantially linear region wherein a major change in compression deflection relates to an anatomically insignificant change in compressive load, with respect to which the mentioned anatomically insignificant change in compressive load relates to a wrap pressure lying in the above-referred-to range of about 0.3- to about 0.7-psi.

Accordingly, while a preferred and best-mode embodiment, and certain modifications thereof, of the structure and methodology of the present invention have been illustrated and described herein, we appreciate that other variations and modifications may be made by those skilled in the art which will come well within the scope and spirit of the present invention.

We claim:

1. A dynamic-response bandaging system comprising
a layered, dynamic-response, anatomical bandaging expanse having inner and outer sides, and including
a moisture-wicking fabric layer having an anatomy-facing side forming the inner side of said expanse applicable directly to, and in contact with, the anatomy,
a dynamic-response, low-rebound, acceleration-rate-rate-sensitive, anatomical-pressure-applying, viscoelastic foam layer joined through a flexible adhesive to said moisture-wicking fabric layer on the opposite side thereof relative to its said anatomy-facing side,
a gas-permeable, moisture-resistant, abrasion-inhibiting fabric layer joined through a flexible adhesive to said viscoelastic foam layer on the opposite side thereof relative to said moisture-wicking fabric layer,
a polyurethane foam layer joined to said abrasion-inhibiting fabric layer on the opposite side thereof relative to said viscoelastic foam layer, and
a pile-portion fabric layer of hook-and-pile material forming the outer side of said expanse joined to said polyurethane foam layer on the opposite side thereof relative to said abrasion-inhibiting fabric layer.

2. The bandaging system of claim 1, wherein said moisture-wicking fabric layer is a tricot fabric layer.

3. The bandaging system of claim 1, wherein each of said flexible adhesives is takes the form of a gas-permeable, moisture-resistant, non-latex adhesive.

4. The bandaging system of claim 1, wherein said viscoelastic foam layer possesses a compressive-load versus compression-deflection behavior characterized by a curve having a substantially linear region in which a major change in compression deflection relates to an anatomically insignificant change in compressive load.

5. The bandaging system of claim 1 which further comprises expanse-cooperative, dynamic-response, flexible, splinting structure including a flexible splint body having an inner side detachably joinable through an affixed hook portion of hook-and-pile structure to said pile-portion fabric layer in said expanse.

6. The bandaging structure of claim 5, wherein said splint body takes the form of a thin, planar, blade-like structure possessing at least one preferential, in-plane bending axis.

7. The bandaging structure of claim 1 which further comprises expanse-cooperative, dynamic-response, flexible, composite, splinting structure including at least a pair of elongate, partially overlapping, flexible splint bodies each having inner and outer sides, with each inner side of each said splint body carrying an affixed hook portion of hook-and-pile structure, and each outer side of each said splint body carrying an affixed pile portion of hook-and-pile structure, one of said splint bodies having its inner side detachably joined to said pile-portion fabric layer, and the other splint body having its inner side joined both (a) to the outer side of said one splint body through the pile portion of hook-and-pile structure affixed to that outer side, and (b) to said pile-portion fabric layer in said expanse.

8. The bandaging structure of claim 7, wherein each said splint body takes the form of a thin, planar, blade-like structure possessing at least one preferential, in-plane bending axis.

9. The bandaging system of claim 1, wherein said expanse further includes spaced, opposite edges, and which is deployable in tension as an overlapping-edge wrap extending around an anatomical limb, and which further comprises an elongate, expanse-edge-attachable/removable, dynamically-responsive, wrap-closure tensioning structure including (a) a pair of spaced, opposite-end, hook-and-pile hook-portion end components adapted for quick attach/detach connection to the outside of said pile-portion fabric layer on opposite sides of an expanse-wrap edge-overlap, and (b) an elongate elastomer bridge extending between and joined to said end components designed, elastomerically and under user-adjustable tension, to span such an expanse-wrap edge-overlap under circumstances with the expanse in an operative, limb-wrapping condition.

10. Dynamic-response anatomical bandaging methodology comprising
placing a dynamic-response, anatomical bandaging expanse as a wrap around a selected portion of an anatomical limb to form a wrapped portion of the limb, and
in relation to and as a consequence of said placing, applying, in accordance with self-compensating response occurring per se within the structure of the placed expanse, dynamically evenized wrap pressure to the wrapped portion of the limb, with such wrap pressure, under all dynamic circumstances with the expanse in place, exceeding that of static fluid pressure in the wrapped limb portion, but being less than that which would block venus-return blood flow in that limb portion.

11. The methodology of claim 10, wherein the mentioned wrap pressure lies in the range of about 0.3- to about 0.7-psi.

12. The methodology of claim 10 which further comprises, before, and to accommodate, said placing, providing a dynamic-response bandaging expanse which is characterized by including a dynamic-response, viscoelastic foam layer formed of a material which exhibits a compressive-load versus compression-deflection behavior characterized by a curve having a substantially linear region wherein a major change in compression deflection relates to an anatomically insignificant change in compressive load.

13. The methodology of claim 12, wherein the mentioned anatomically insignificant change in compressive load relates to a wrap pressure lying in the range of about 0.3- to about 0.7-psi.
